(19) United States
(12) Patent Application Publication
(10) Pub. No.: US 2022/0031171 A1
(43) Pub. Date: Feb. 3, 2022

(54) POPULATION MALADY IDENTIFICATION WITH A WEARABLE GLUCOSE MONITORING DEVICE

(71) Applicant: DexCom, Inc., San Diego, CA (US)

(72) Inventors: Joost van der Linden, San Diego, CA (US); Anna Claire Harley-Trochimczyk, San Diego, CA (US)

(21) Appl. No.: 17/385,529

(22) Filed: Jul. 26, 2021

Related U.S. Application Data
(60) Provisional application No. 63/058,253, filed on Jul. 29, 2020.

(51) Int. Cl. A61B 5/01; A61B 5/00; A61B 5/145

(52) U.S. Cl. CPC A61B 5/01; A61B 5/6801; A61B 5/746; A61B 5/7264; A61B 5/742; A61B 5/14532

(57) ABSTRACT

Population malady identification with a wearable glucose monitoring device is described. A malady identification system obtains temperature measurements that are produced by wearable glucose monitoring devices worn by users of a user population. The malady identification system further obtains location data describing locations of the users and associates each of the temperature measurements with a respective location. The malady identification system utilizes identification logic (e.g., one or more machine learning models) to identify presence of a malady in the users at one or more of the locations based on the temperature measurements and the location data. The malady identification system generates a communication for notifying at least one of the users about the presence of the malady.

### RELATED APPLICATION

This application claims the benefit of U.S. Provisional Patent Application No. 63/058,253, filed Jul. 29, 2020, and titled “Population Malady Identification with a Wearable Glucose Monitoring Device,” the entire disclosure of which is hereby incorporated by reference.

### BACKGROUND

Maladies, such as diseases like influenza and coronavirus, can have wide-ranging and severe effects on a population. For example, a given malady may have health-related impacts on the population, requiring at least some form of treatment for persons affected with the malady. Additionally, some maladies may have severe economic impacts, crippling sectors of the world and local economies for a variety of reasons, such as fear of the malady, rules and regulations enacted in response to spread of the malady (e.g., quarantine, curfews, and/or closure of certain businesses), and so forth. The negative effects of such maladies can be reduced, however, by adoption of mitigating behaviors, such as social distancing, increased handwashing or sanitizing, increased cleaning of shared spaces, and wearing facial coverings, to name just a few.

The effectiveness of those behaviors to actually mitigate a malady depends not only on the extent to which the behaviors are adopted but also on a timeliness of the adoption. Widespread adoption of mitigating behaviors earlier in the course of a malady, can curtail the negative impacts of a malady to a greater degree than if those behaviors are adopted later. In addition, some persons may be at a higher risk than others of experiencing severe adverse effects if they become infected. One example of persons that may be at higher risk than others are persons with diabetes—a given malady may cause more severe adverse effects, potentially life threatening, in a person with diabetes as compared to a person who does not have diabetes. Earlier adoption of mitigating behaviors by those higher-risk persons enables them to limit their exposure to a malady and/or take precautions that prevent contraction of the malady, even if exposed to it.

An indicator of many maladies is an increased temperature of a person having a malady, where increased is relative to an established “normal” temperature such as 98.6° F. In the real world, though, persons generally do not “take their temperature” until they feel ill, such that after a person begins to feel ill, he, she, or a care giver may obtain a thermometer to measure the person's temperature and/or may travel to a doctors' office where his or her temperature is measured. By this time, however, the person may have been infected with the malady for enough time to unknowingly expose other persons to the malady. Early during a malady's onset, many persons of a population may unknowingly have and expose others to the malady due to the time delay between contracting the malady and feeling ill enough to measure their temperatures. Due at least in part to this time delay conventional approaches for identifying a malady using temperatures may not be suitable to prevent the spread of the malady, and the malady may therefore have severe impacts on the population and also on persons who are at higher risk for experiencing severe adverse effects.

### SUMMARY

To overcome these problems, population malady identification with a wearable glucose monitoring device is leveraged. A malady identification system obtains temperature measurements that are produced by wearable glucose monitoring devices worn by users of a user population. The malady identification system further obtains location data describing locations of the users and associates the temperature measurements with a respective location. The malady identification system utilizes identification logic (e.g., one or more machine learning models) to identify presence of a malady in the users at one or more of the locations based on the temperature measurements and the location data. The malady identification system generates a communication for notifying at least one of the users about the presence of the malady.

One aspect is a method comprising: obtaining temperature measurements produced by wearable glucose monitoring devices worn by users of a user population; obtaining location data describing locations of the users and associating each of the temperature measurements with a respective location; identifying presence of a malady in the users at one or more of the locations based on the temperature measurements and the location data; and notifying at least one of the users about the presence of the malady.

In the above method, identifying the presence of the malady includes processing the temperature measurements and the location data, in part, using one or more machine learning models, the one or more machine learning models generated based on historical temperature measurements of the user population and historical data describing presence of one or more maladies in the user population. In the above method, the wearable glucose monitoring devices include at least one continuous glucose monitoring (CGM) system. In the above method, the identifying is further based on glucose measurements produced by the wearable glucose monitoring devices worn by the users of the user population.

In the above method, identifying the presence of the malady includes processing the temperature measurements, the glucose measurements, and the location data, in part, using one or more machine learning models, the one or more machine learning models generated based on historical temperature and glucose measurements of the user population and historical data describing presence of one or more maladies in the user population.

The above method further comprises notifying at least one third party about the presence of the malady. In the above method, the at least one third party includes at least one of: a public health organization, a governmental organization, a school district, a health care facility, a news source, a telemedicine service, or a data partner with a glucose monitoring platform that corresponds to the wearable glucose monitoring devices. In the above method, the users of the user population have user profiles with a glucose monitoring platform.

In the above method, notifying at least one user about the presence of the malady includes: generating a heat map that visually differentiates severity of the malady across user populations at different locations; and causing display of the heat map on a display device of a computing device associated with the at least one user. In the above method, notifying at least one user about the presence of the malady includes: generating an alert having information about the presence of the malady; and causing output of the alert via a computing device associated with the at least one user. In the above method, causing output of the alert comprises causing display of the alert via a display device of the computing device.

Another aspect is a system comprising: at least one processor; and memory having instructions stored thereon that are executable by the at least one processor to perform operations including: obtaining temperature measurements produced by wearable glucose monitoring devices worn by users of a user population; obtaining location data describing locations of the users and associating the temperature measurements with a respective location; identifying presence of a malady in the users at one or more of the locations based on the temperature measurements and the location data; and notifying at least one of the users about the presence of the malady.

The above system further comprises one or more machine learning models configured to identify the presence of the malady by processing the temperature measurements and the location data, the one or more machine learning models generated based on historical temperature measurements of the user population and historical data describing presence of one or more maladies in the user population.

In the above system, the wearable glucose monitoring devices include at least one continuous glucose monitoring (CGM) system. In the above system, the identifying is further based on glucose measurements produced by the wearable glucose monitoring devices worn by the users of the user population. The above system further comprises one or more machine learning models configured to identify the presence of the malady by processing the temperature measurements, the glucose measurements, and the location data, the one or more machine learning models generated based on historical temperature and glucose measurements of the user population and historical data describing presence of one or more maladies in the user population.

In the above system, the operations further include notifying at least one third party about the presence of the malady. In the above system, the at least one third party includes at least one of: a public health organization, a governmental organization, a school district, a health care facility, a news source, a telemedicine service, or a data partner with a glucose monitoring platform that corresponds to the wearable glucose monitoring devices. In the above system, the users of the user population have user profiles with a glucose monitoring platform.

In the above system, notifying at least one user about the presence of the malady includes: generating a heat map that visually differentiates severity of the malady across user populations at different locations; and causing display of the heat map on a display device of a computing device associated with the at least one user. In the above system, notifying at least one user about the presence of the malady includes: generating an alert having information about the presence of the malady; and causing output of the alert via a computing device associated with the at least one user. In the above system, causing output of the alert comprises causing display of the alert via a display device of the computing device.

Another aspect is one or more non-transitory computer-readable storage media having instructions stored thereon that are executable by one or more processors of at least one computing device to cause the at least one computing device to perform operations comprising: obtaining temperature measurements produced by wearable glucose monitoring devices worn by users of a user population; obtaining location data describing locations of the users and associating the temperature measurements with a respective location; identifying presence of a malady in the users at one or more of the locations based on the temperature measurements and the location data; and notifying at least one of the users about the presence of the malady.

In the above storage media, identifying the presence of the malady includes processing the temperature measurements and the location data, in part, using one or more machine learning models, the one or more machine learning models generated based on historical temperature measurements of the user population and historical data describing presence of one or more maladies in the user population. In the above storage media, the wearable glucose monitoring devices include at least one continuous glucose monitoring (CGM) system. In the above storage media, the identifying is further based on glucose measurements produced by the wearable glucose monitoring devices worn by the users of the user population.

In the above storage media, identifying the presence of the malady includes processing the temperature measurements, the glucose measurements, and the location data, in part, using one or more machine learning models, the one or more machine learning models generated based on historical temperature and glucose measurements of the user population and historical data describing presence of one or more maladies in the user population. In the above storage media, the operations further include notifying at least one third party about the presence of the malady.

In the above storage media, the at least one third party includes at least one of: a public health organization, a governmental organization, a school district, a health care facility, a news source, a telemedicine service, or a data partner with a glucose monitoring platform that corresponds to the wearable glucose monitoring devices. In the above storage media, the users of the user population have user profiles with a glucose monitoring platform. In the above storage media, notifying at least one user about the presence of the malady includes: generating a heat map that visually differentiates severity of the malady across user populations at different locations; and causing display of the heat map on a display device of a computing device associated with the at least one user.

In the above storage media, notifying at least one user about the presence of the malady includes: generating an alert having information about the presence of the malady; and causing output of the alert via a computing device associated with the at least one user. In the above storage media, causing output of the alert comprises causing display of the alert via a display device of the computing device.

Another aspect is an apparatus comprising: means for obtaining temperature measurements produced by wearable glucose monitoring devices worn by users of a user population; means for obtaining location data describing locations of the users and associating each of the temperature measurements with a respective location; means for identifying presence of a malady in the users at one or more of the locations based on the temperature measurements and the location data; and means for notifying at least one of the users about the presence of the malady.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example implementation that is operable to employ techniques described herein.

FIG. 2 depicts an example of the wearable glucose monitoring device of FIG. 1 in greater detail.

FIG. 3 depicts an example implementation in which data collected from the wearable glucose monitoring device, including temperature measurements, is routed to different systems in connection with population malady identification.

FIG. 4 depicts an example implementation of a user interface displayed for presenting information associated with population malady identification.

FIG. 5 depicts an example implementation of information presented in association with population malady identification via a user interface.

FIG. 6 depicts an example implementation of a user interface displayed for presenting a notification associated with population malady identification.

FIG. 7 depicts an example implementation of user interfaces displayed for presenting information associated with population malady identification at a selected location.

FIG. 8 depicts a procedure in an example implementation in which presence of a malady is identified in users at one or more locations based on temperature measurements obtained from wearable glucose monitoring devices.

FIG. 9 illustrates an example system including various components of an example device that can be implemented as any type of computing device as described and/or utilized with reference to FIGS. 1-8 to implement embodiments of the techniques described herein.

### DETAILED DESCRIPTION

### Overview

An indicator of many maladies is an increased temperature of a person having a malady, where increased is relative to an established “normal” temperature such as 98.6° F. In the real world, though, persons generally do not “take their temperature” until they feel ill—they do not generally monitor their temperature continuously in substantially real-time. By the time an ill person's temperature is actually taken, the person may have been infected with the malady for enough time to unknowingly expose other persons. Early during a malady's onset, many persons of a population may unknowingly have and expose others to the malady due to the time delay between contracting the malady and feeling ill enough to measure their temperatures. Due at least in part to this time delay, conventional approaches for identifying a malady using temperatures may not be suitable to prevent the spread of the malady, and the malady may therefore have severe impacts on the population and also on persons who are at higher risk for experiencing severe adverse effects. In addition, there are a variety of factors that can affect an individual person's temperature. For instance, exercise and weather can affect an individual's temperature, such that the individual's temperature may correspond to temperatures that generally indicate the presence or absence of a malady—even though the exercise or weather is the cause of the temperature and not a malady. To this end, there may be too much noise in an individual person's temperature measurements to make temperature suitable, at an individual level, for malady identification.

To overcome these problems, population malady identification with a wearable glucose monitoring device is leveraged. Unlike conventional temperature measurement approaches, a wearable glucose monitoring device configured with a temperature sensor may produce temperature measurements of a person continuously (e.g., at predetermined time intervals) and in real-time, since the glucose monitoring device is configured for continuous wear by the person over a time period. By producing real-time temperature measurements of the person, without interaction of the person or another to intentionally take the person's temperature, the wearable glucose monitoring device captures changes to the person's temperature as those changes occur. To the extent that a significant subset of persons in a geographic region may wear a wearable glucose monitoring device configured with a temperature sensor, the wearable glucose monitoring devices worn by the subset may produce temperature measurements in real-time, such that changes in the temperature measurements across the subset can be identified, e.g., by identification logic such as a machine learning model.

In one or more implementations, a malady identification system obtains those temperature measurements produced by wearable glucose monitoring devices worn by users of a user population. The malady identification system further obtains location data describing locations of the users and associates each of the temperature measurements with a respective location. For example, the identification system obtains the location data from user profiles of the users or from location data (e.g., global positioning system (GPS) coordinates) packaged with the temperature measurements, e.g., by mobile devices of the users.

The malady identification system utilizes identification logic (e.g., one or more machine learning models) to identify presence of a malady in the users at one or more of the locations based on the temperature measurements and the location data. It is to be appreciated that the number of temperature measurements obtained for a population of users is too numerous for a human to practically process, e.g., to identify meaningful patterns in those temperature measurements (e.g., a rise of average temperatures at a location that surpass a threshold temperature) across the population and/or in specific locations. By contrast, however, the identification logic is configured to process numbers of temperature measurements and in an amount of time that is practically impossible for any human.

Once the identification logic identifies the presence or absence of a malady, the malady identification system may generate a communication for notifying at least one of the users about the presence of the malady. Additionally or alternately, the malady identification system may generate communications for notifying third parties, such as public health organizations, governmental agencies, school districts, and so on. By measuring temperatures at a population level in real-time and notifying about the presence of a malady, the malady identification system can provide information about the presence of a malady earlier than conventional approaches. As a result, persons may be able to adopt mitigating behaviors earlier, which may be effective to avoid or at least reduce many of the negative effects of maladies.

In the following discussion, an example environment is first described that may employ the techniques described herein. Example implementation details and procedures are then described which may be performed in the example environment as well as other environments. Performance of the example procedures is not limited to the example environment and the example environment is not limited to performance of the example procedures.

### Example Environment

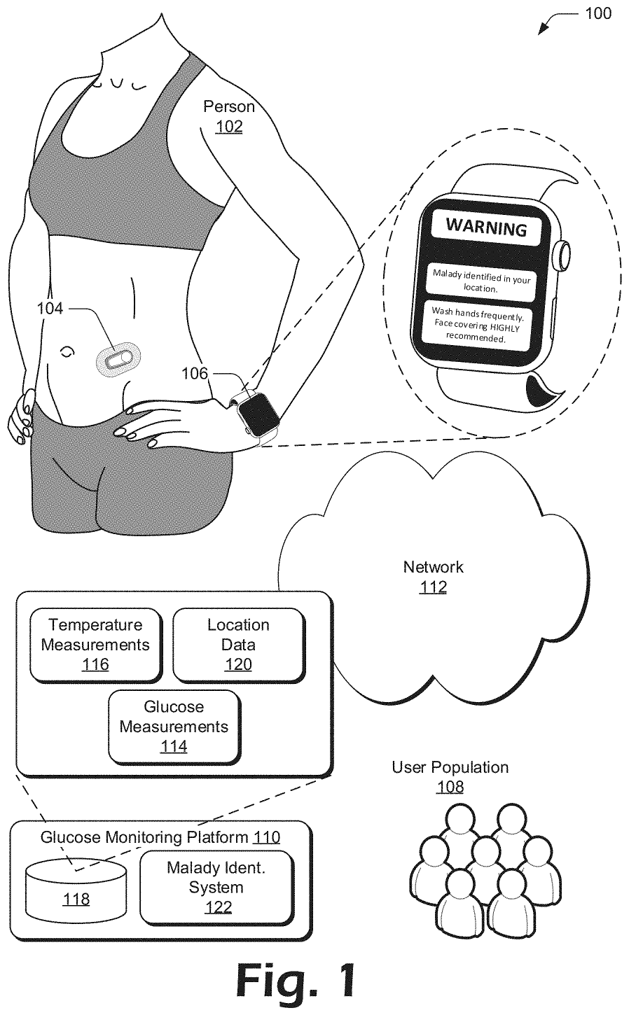

FIG. 1 is an illustration of an environment 100 in an example implementation that is operable to employ population malady identification with a wearable glucose monitoring device as described herein. The illustrated environment 100 includes person 102, who is depicted wearing a wearable glucose monitoring device 104 and computing device 106. The illustrated environment 100 also includes other users in a user population 108 that wear the wearable glucose monitoring device 104, and glucose monitoring platform 110. The wearable glucose monitoring device 104, computing device 106, user population 108, and glucose monitoring platform 110 are communicatively coupled, including via a network 112.

Alternately or additionally, the wearable glucose monitoring device 104 and the computing device 106 may be communicatively coupled in other ways, such as using one or more wireless communication protocols or techniques. By way of example, the wearable glucose monitoring device 104 and the computing device 106 may communicate with one another using one or more of Bluetooth (e.g., Bluetooth Low Energy links), near-field communication (NFC), 5G, and so forth.

In accordance with the described techniques, the wearable glucose monitoring device 104 is configured to provide measurements of person 102's blood glucose and also measurements of the person 102's temperature. The wearable glucose monitoring device 104 may be configured with a glucose sensor, for instance, that continuously detects analytes indicative of the person 102's glucose and enables generation of glucose measurements. In the illustrated environment 100 and throughout the detailed description these measurements are represented as glucose measurements 114. The wearable glucose monitoring device 104 may also be configured with a temperature sensor, for instance, such as a thermocouple that continuously measures a temperature-dependent voltage as a result of a thermoelectric effect. The measured voltage can be interpreted as a temperature (e.g., of the person 102). In the illustrated environment 100 and throughout the detailed description those measurements are represented as temperature measurements 116.

In one or more implementations, the wearable glucose monitoring device 104 is a continuous glucose monitoring (CGM) system. As used herein, the term “continuous” used in connection with glucose monitoring may refer to an ability of a device to produce measurements substantially continuously, such that the device may be configured to produce the glucose measurements 114 at intervals of time (e.g., every hour, every 30 minutes, every 5 minutes, and so forth), responsive to establishing a communicative coupling with a different device (e.g., when a computing device establishes a wireless connection with the wearable glucose monitoring device 104 to retrieve one or more of the measurements), and so forth

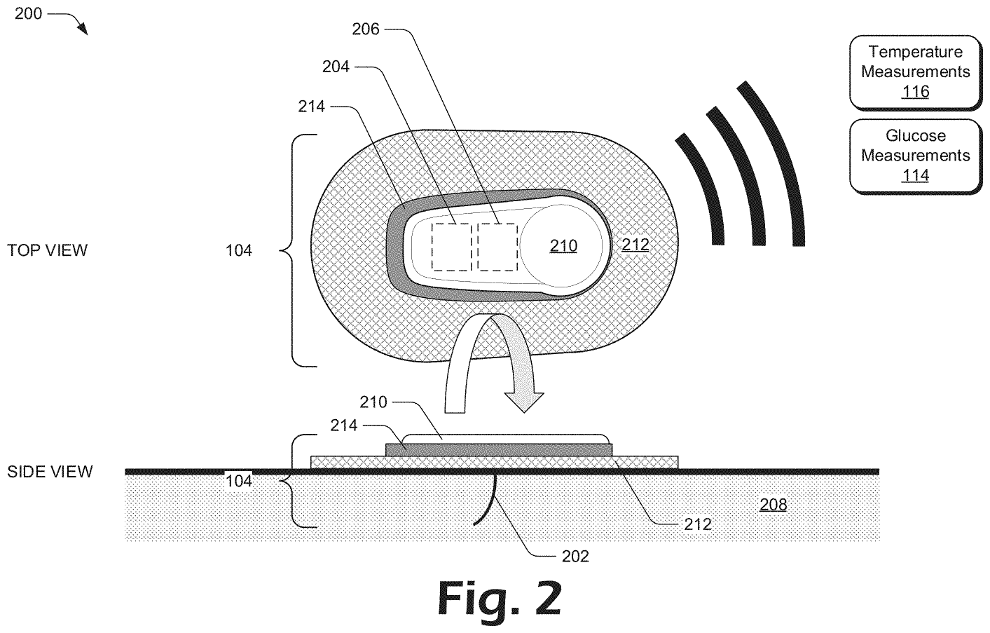

In a similar fashion, the wearable glucose monitoring device 104 may be configured to produce the temperature measurements 116 substantially continuously, such as to enable the temperature measurements 116 to be produced at intervals of time (e.g., every 5 minutes, every minute, every 30 seconds, and so on), responsive to establishing a communicative coupling with a different device, and so forth. In one or more implementations, a rate which the temperature measurements 116 are produced is more frequent than a rate at which the glucose measurements 114 are produced, e.g., the temperature measurements 116 are produced every 30 seconds and the glucose measurements 114 are produced every 5 minutes. This functionality along with further aspects of the wearable glucose monitoring device 104's configuration are discussed in more detail in relation to FIG. 2.

Additionally, the wearable glucose monitoring device 104 transmits the glucose measurements 114 and the temperature measurements 116 to the computing device 106, such as via a wireless connection. The wearable glucose monitoring device 104 may communicate these measurements in real-time, e.g., as they are produced using a glucose sensor and a temperature sensor. Alternately or in addition, the wearable glucose monitoring device 104 may communicate the glucose measurements 114 and the temperature measurements 116 to the computing device 106 at set time intervals. For example, the wearable glucose monitoring device 104 may be configured to communicate the glucose measurements 114 to the computing device 106 every five minutes (as they are being produced) and to communicate the temperature measurements 116 every 30 seconds (as they are being produced) or once a day (as part of a “data dump” at a predetermined interval of time).

Certainly, an interval at which the glucose measurements 114 and an interval at which the temperature measurements 116 are communicated may be different from the examples above without departing from the spirit or scope of the described techniques. The measurements may be communicated by the wearable glucose monitoring device 104 to the computing device 106 according to other bases in accordance with the described techniques, such as based on a request from the computing device 106. Regardless, the computing device 106 may maintain the glucose measurements 114 and/or the temperature measurements 116 of the person 102 at least temporarily, e.g., in computer-readable storage media of the computing device 106.

Although illustrated as a wearable device (e.g., a smart watch), the computing device 106 may be configured in a variety of ways without departing from the spirit or scope of the described techniques. By way of example and not limitation, the computing device 106 may be configured as a different type of mobile device (e.g., a mobile phone or tablet device). In one or more implementations, the computing device 106 may be configured as a dedicated device associated with the glucose monitoring platform 110, e.g., with functionality to obtain the glucose measurements 114 from the wearable glucose monitoring device 104, perform various computations in relation to the glucose measurements 114, display information related to the glucose measurements 114 and the glucose monitoring platform 110, communicate the glucose measurements 114 to the glucose monitoring platform 110, and so forth. In contrast to implementations where the computing device 106 is configured as a mobile phone, however, the computing device 106 may not include some functionality available with mobile phone or wearable configurations when configured as a dedicated device, such as the ability to make phone calls, camera functionality, the ability to utilize social networking applications, and so on.

Additionally, the computing device 106 may be representative of more than one device in accordance with the described techniques. In one or more scenarios, for instance, the computing device 106 may correspond to both a wearable device (e.g., a smart watch) and a mobile phone. In such scenarios, both of these devices may be capable of performing at least some of the same operations, such as to receive the glucose measurements 114 and the temperature measurements 116 from the wearable glucose monitoring device 104, communicate them via the network 112 to the glucose monitoring platform 110, display information related to the glucose measurements 114 and the temperature measurements 116, and so forth. Alternately or in addition, different devices may have different capabilities that other devices do not have or that are limited through computing instructions to specified devices.

In the scenario where the computing device 106 corresponds to a separate smart watch and a mobile phone, for instance, the smart watch may be configured with various sensors and functionality to measure a variety of physiological markers (e.g., heartrate, breathing, rate of blood flow, and so on) and activities (e.g., steps) of the person 102. In this scenario, the mobile phone may not be configured with these sensors and functionality, or it may include a limited amount of that functionality—although in other scenarios a mobile phone may be able to provide the same functionality. Continuing with this particular scenario, the mobile phone may have capabilities that the smart watch does not have, such as a camera to capture images of meals used to predict future glucose levels and an amount of computing resources (e.g., battery and processing speed) that enables the mobile phone to more efficiently carry out computations in relation to the glucose measurements 114 and the temperature measurements 116. Even in scenarios where a smart watch is capable of carrying out such computations, computing instructions may limit performance of those computations to the mobile phone so as not to burden both devices and to utilize available resources efficiently. To this extent, the computing device 106 may be configured in different ways and represent different numbers of devices than discussed herein without departing from the spirit and scope of the described techniques.

As mentioned above, the computing device 106 communicates the glucose measurements 114 and the temperature measurements 116 to the glucose monitoring platform 110. In the illustrated environment 100, the glucose measurements 114 and the temperature measurements 116 are shown stored in storage device 118 of the glucose monitoring platform 110 along with location data 120. The storage device 118 may represent one or more databases and also other types of storage capable of storing the glucose measurements 114, the temperature measurements 116, and the location data 120.

The storage device 118 may also store a variety of other data. In accordance with the described techniques, for instance, the person 102 corresponds to a user of at least the glucose monitoring platform 110 and may also be a user of one or more other, third party service providers. To this end, the person 102 may be associated with a username and be required, at some time, to provide authentication information (e.g., password or biometric data) to access the glucose monitoring platform 110 using the username. This information, along with other information about the user, may be maintained in the storage device 118, including, for example, demographic information describing the person 102, information about a health care provider, payment information, prescription information, determined health indicators, user preferences, account information for other service provider systems (e.g., a service provider associated with a wearable, social networking systems, telemedicine services, and so on), and so forth.

The storage device 118 also maintains data of the other users in the user population 108. Given this, the glucose measurements 114 and the temperature measurements 116 in the storage device 118 include the glucose and temperature measurements from glucose and temperature sensors of the wearable glucose monitoring device 104 worn by the person 102 and also include glucose and temperature measurements from glucose and temperature sensors of glucose monitoring devices worn by persons corresponding to the other users in the user population 108. It follows also that the glucose measurements 114 and temperature measurements 116 of these other users are communicated by their respective devices via the network 112 to the glucose monitoring platform 110 and that these other users have respective user profiles with the glucose monitoring platform 110.

In the illustrated example, the glucose monitoring platform 110 includes malady identification system 122. The malady identification system 122 is configured to process at least the temperature measurements 116 and the location data 120 to identify presence of a malady among a population of users in a location, such as to identify an outbreak of coronavirus disease in a geographic region, e.g., a country, state, county, city, zip code, voting district, or school district, to name just a few. Based on identification of a malady among a population, the malady identification system 122 may provide notifications in relation to the identification, such as alerts, recommendations, “heat” maps, or other information based on the predictions. For instance, the malady identification system 122 may provide the notifications to the person 102 (e.g., via the computing device 106), to a public health organization, and so forth.

Although depicted as part of a separate apparatus from the computing device 106, portions or an entirety of the malady identification system 122 may alternately or additionally be implemented at the computing device 106, e.g., a malady identification app. The malady identification system 122 may also identify a malady among a population of users at a location using additional data, such as by using the glucose measurements 114. In the context of measuring glucose and temperature, e.g., continuously, and obtaining data describing such measurements, consider the following discussion of FIG. 2.

FIG. 2 depicts an example 200 of an implementation of the wearable glucose monitoring device 104 of FIG. 1 in greater detail. In particular, the illustrated example 200 includes a top view and a corresponding side view of the wearable glucose monitoring device 104. It is to be appreciated that the wearable glucose monitoring device 104 may vary in implementation from the following discussion in various ways without departing from the spirit or scope of the described techniques.

In this example 200, the wearable glucose monitoring device 104 is illustrated to include a glucose sensor 202, a temperature sensor 204, and a sensor module 206. Here, the glucose sensor 202 is depicted in the side view having been inserted subcutaneously into skin 208, e.g., of the person 102. The temperature sensor 204 and the sensor module 206 are depicted in the top view as dashed rectangles. The wearable glucose monitoring device 104 also includes a transmitter 210 in the illustrated example 200. Use of the dashed rectangles for the temperature sensor 204 and the sensor module 206 indicate that they may be housed or otherwise implemented within a housing of the transmitter 210. In this example 200, the wearable glucose monitoring device 104 further includes adhesive pad 212 and attachment mechanism 214.

In operation, the glucose sensor 202, the adhesive pad 212, and the attachment mechanism 214 may be assembled to form an application assembly, where the application assembly is configured to be applied to the skin 208 so that the glucose sensor 202 is subcutaneously inserted as depicted. In such scenarios, the transmitter 210 may be attached to the assembly after application to the skin 208 via the attachment mechanism 214. Additionally or alternately, the transmitter 210 may be incorporated as part of the application assembly, such that the glucose sensor 202, the adhesive pad 212, the attachment mechanism 214, and the transmitter 210 (with the temperature sensor 204 and the sensor module 206) can all be applied at once to the skin 208. Additionally or alternately, the temperature sensor 204 may be disposed with the glucose sensor 202 so that it is included with an application assembly, which may include the transmitter 210 in some configurations and may not include the transmitter 210 in other configurations (e.g., the transmitter 210 may be attached to the application assembly after application). In one or more implementations, this application assembly is applied to the skin 208 using a separate sensor applicator (not shown). In one or more implementations, the application assembly may be removed by peeling the adhesive pad 212 off of the skin 208. It is to be appreciated that the wearable glucose monitoring device 104 and its various components as illustrated are simply one example form factor, and the wearable glucose monitoring device 104 and its components may have different form factors without departing from the spirit or scope of the described techniques.

In operation, the glucose sensor 202 and the temperature sensor 204 are communicatively coupled to the sensor module 206 via at least one communication channel, which can be a wireless connection or a wired connection. Communications from the glucose sensor 202 and the temperature sensor 204 to the sensor module 206 or from the sensor module 206 to the glucose sensor 202 and the temperature sensor 204 can be implemented actively or passively and these communications can be continuous (e.g., analog) or discrete (e.g., digital).

The glucose sensor 202 may be a device, a molecule, and/or a chemical which changes or causes a change in response to an event which is at least partially independent of the glucose sensor 202. The sensor module 206 is implemented to receive indications of changes to the glucose sensor 202 or caused by the glucose sensor 202. For example, the glucose sensor 202 can include glucose oxidase which reacts with glucose and oxygen to form hydrogen peroxide that is electrochemically detectable by the sensor module 206 which may include an electrode. In this example, the glucose sensor 202 may be configured as or include a glucose sensor configured to detect analytes in blood or interstitial fluid that are indicative of glucose level using one or more measurement techniques. In one or more implementations, the glucose sensor 202 may also be configured to detect analytes in the blood or the interstitial fluid that are indicative of other markers, such as lactate levels. Additionally or alternately, the wearable glucose monitoring device 104 may include additional sensors to the glucose sensor 202 to detect those analytes indicative of the other markers.

The temperature sensor 204 is configured to detect conditions that can be used to determine temperature measurements, e.g., of the person 102. For instance, the temperature sensor 204 may be configured as a thermocouple that continuously measures a temperature-dependent voltage as a result of a thermoelectric effect. The sensor module 206 may be configured to interpret the measured voltage as a temperature (e.g., of the person 102), and produce the temperature measurements 116. Alternately or additionally, the temperature sensor 204 can include or utilize a first and second electrical conductor and the sensor module 206 can electrically detect changes in electric potential across the first and second electrical conductor of the temperature sensor 204. In this example, the sensor module 206 and the temperature sensor 204 are configured as a thermocouple such that the changes in electric potential correspond to temperature changes, which the sensor module 206 may be configured to use to produce the temperature measurements 116. It is to be appreciated that the temperature sensor 204 and the sensor module 206 may be configured in a variety of ways to detect a temperature of the person 102 and to produce the temperature measurements 116 to indicate the person 102's temperature.

In some examples, the sensor module 206 and the sensors of the wearable glucose monitoring device 104 are configured to detect a single analyte, e.g., glucose. In other examples, the sensor module 206 and the sensors of the wearable glucose monitoring device 104 are configured to detect multiple analytes, e.g., sodium, potassium, carbon dioxide, testosterone, lactate, insulin, and glucose. Alternately or additionally, the wearable glucose monitoring device 104 may include multiple sensors to detect not only one or more analytes (e.g., sodium, potassium, carbon dioxide, testosterone, lactate, insulin, and glucose) but also one or more environmental conditions (e.g., temperature of the person 102, temperature of the environment where there person 102 is located). Thus, the sensor module 206, the glucose sensor 202, and the temperature sensor 204 (as well as any additional sensors) may detect the presence of one or more analytes, the absence of one or more analytes, a temperature of the person 102, and/or changes in one or more environmental conditions.

In one or more implementations, the sensor module 206 may include a processor and memory (not shown). The sensor module 206, by leveraging the processor, may generate the temperature measurements 116 based on the communications with the temperature sensor 204, e.g., that are indicative of the above-discussed interpretations. Similarly, the sensor module 206, by leveraging the processor, may generate the glucose measurements 114 based on the communications with the glucose sensor 202, e.g., that are indicative of the above-discussed changes. Based on these communications with the glucose sensor 202 and the temperature sensor 204, the sensor module 206 may be further configured to generate streams of the temperature measurements 116 and the glucose measurements 114. These streams may comprise communicable packages of data that include at least one glucose measurement 114 or at least one temperature measurement 116.

As noted above, the sensor module 206 and the sensors may operate to produce the glucose measurements 114 and the temperature measurements 116 at different intervals of time. For example, the sensor module 206 and the temperature sensor 204 may be operable to produce a temperature measurement 116 (e.g., of the person 102) at a first interval of time, e.g., every 30 seconds. By way of contrast, the sensor module 206 and the glucose sensor 202 may be operable to produce a glucose measurement 114 at a second interval of time that is different from the first interval of time, e.g., every 5 minutes. Certainly, intervals of time at which the glucose measurements 114 and temperature measurements 116 are produced using the glucose sensor 202 and the temperature sensor 204 may vary from those discussed above in accordance with the described techniques. For example, the glucose measurements 114 and temperature measurements 116 may be produced at a same interval of time in at least one implementation, such that there is a one-to-one relationship between the glucose measurements 114 and the temperature measurements 116.

In addition to being produced at different intervals of time, the glucose measurements 114 and the temperature measurements 116 may also be communicated to one or more computing devices at different intervals of time. For example, the transmitter 210 may be configured to transmit the glucose measurements 114 to a computing device at a first transmission interval, e.g., every 5 minutes. In one or more implementations, the transmission interval of the glucose measurements 114 is the same as an interval at which the glucose measurements 114 are produced, e.g., every 5 minutes. In this way, each time an individual glucose measurement 114 is produced, the transmitter 210 may communicate the individual glucose measurement 114 to a computing device. Additionally or alternately, a plurality of the glucose measurements 114 may be stored by storage of the wearable glucose monitoring device 104, and the transmitter 210 may communicate the plurality of glucose measurements 114 (or a subset of them) to a computing device.

With respect to the temperature measurements 116, the transmitter 210 may be configured to communicate them to a computing device at a second transmission interval that is different from the first transmission interval, e.g., once a day. To this end, a plurality of the temperature measurements 116 may be maintained at least temporarily in storage of the wearable glucose monitoring device 104. Those temperature measurements 116 may be communicated by the transmitter 210 to a computing device at a different rate (e.g., once a day) than a rate at which those measurements are produced (e.g., every 30 seconds). By communicating the temperature measurements 116 less frequently than those measurements are produced, the wearable glucose monitoring device 104 may conserve resources (e.g., battery life and computer processing cycles) that may be otherwise used generating and communicating more frequent communications of the temperature measurements 116 to a computing device. In contrast, the transmitter 210 may communicate the glucose measurements 114 substantially as they are produced because the person 102 or a health care provider may use the glucose measurements 114 to make treatment decisions for a health condition (e.g., diabetes). Moreover, such decisions may require the glucose measurements 114 to be timely (i.e., substantially in real-time) in order to avoid adverse effects associated with the health condition, e.g., dysglycemia. Although a transmission interval that is less frequent than the production of the temperature measurements 116 is discussed above, in one or more implementations the transmitter 210 may transmit the temperature measurements 116 to a computing device at a same or similar rate at which those measurements are produced.

The transmitter 210 may also cause transmission to a computing device of additional data packaged with or separate from the glucose measurements 114 and the temperature measurements 116. By way of example, this additional data may include measurements of other analytes, one or more sensor identifiers (e.g., information that uniquely identifies the particular glucose sensor 202 from other glucose sensors), identifiers of other components of the wearable glucose monitoring device 104 (e.g., one or more antennae of the transmitter 210), a sensor status that represents a state of a given sensor (e.g. describing an operation state of the given sensor),

Having considered an example environment and example wearable glucose monitoring device, consider now a discussion of some example details of the techniques for population malady identification with a wearable glucose monitoring device in a digital medium environment in accordance with one or more implementations.

Population Malady Identification

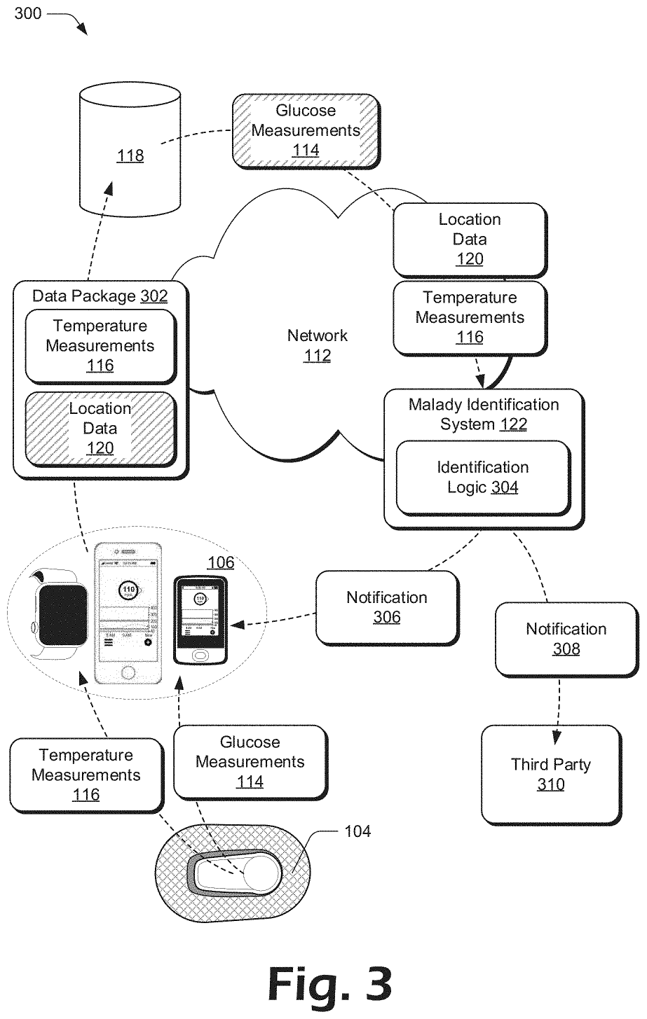

FIG. 3 depicts an example 300 of an implementation in which data collected from the wearable glucose monitoring device, including temperature measurements, is routed to different systems in connection with population malady identification.

The illustrated example 300 includes from FIG. 1 the wearable glucose monitoring device 104 and examples of the computing device 106. The illustrated example 300 also includes the malady identification system 122 and the storage device 118, which, as discussed above, stores the glucose measurements 114 and the temperature measurements 116. In this example 300, the wearable glucose monitoring device 104 is depicted transmitting the glucose measurements 114 and the temperature measurements 116 to the computing device 106. The wearable glucose monitoring device 104 may transmit the glucose measurements 114 and the temperature measurements 116 to the computing device 106 in a variety of ways.

The illustrated example 300 also includes data package 302. Here, the data package 302 includes the temperature measurements 116 and location data 120. The location data 120 is illustrated with hashing to indicate that it is optional—in one or more implementations location data is not communicated with the temperature measurements 116 to the glucose monitoring platform 110. In scenarios where the location data 120 is not communicated with the temperature measurements 116, the location data 120 may simply be maintained in the storage device 118 of the glucose monitoring platform 110, e.g., as a location entered in connection with establishing or updating a user profile of the person 102 with the glucose monitoring platform 110. This is one example of how a temperature measurement 116 of the person 102 may be associated with a location.

In one or more implementations, the location data 120 may be generated by the computing device 106 and packaged in the data package 302 (as illustrated) along with the temperature measurements 116 to describe a location of the person 102, such as a location of the person 102 when the glucose measurements 114 are produced or when the glucose measurements 114 are communicated to the glucose monitoring platform 110. By way of example, the computing device 106 may be configured with suitable hardware and processing resources to determine the location using global positioning system (GPS) coordinates, triangulation approaches that involve communication with wireless access points (e.g., wireless routers or cell phone towers), a combination of GPS and other data received wirelessly, and so forth. In this way, a temperature measurement 116 of the person 102 at a given time can be associated with a location where the person 102 is located—or where the computing device 106 of the person 102 is physically located.

The data package 302 may include different or additional data than illustrated, such as one or more of the glucose measurements 114, any other data produced by the wearable glucose monitoring device 104 and communicated to the computing device 106, and supplemental data produced by the computing device 106 that describes one or more events that correspond (e.g., in time) to one or more of the glucose measurements 114 and/or the temperature measurements 116 (e.g., application usage data, device interaction data, and so forth), to name just a few. In this example 300, the data package 302 is depicted being routed from the computing device 106 to the storage device 118 of the glucose monitoring platform 110. Thus, the computing device 106 may act as an intermediary between the wearable glucose monitoring device 104 and the glucose monitoring platform 110, and the computing device 106 may be configured, for example, as a mobile phone or a smart watch of the user.

Although not depicted in the illustrated example 300, the glucose monitoring platform 110 may process these data packages 302 and cause at least some of the temperature measurements 116 and the location data 120 (when included in the data packages 302) to be stored in the storage device 118. The glucose monitoring platform 110 may also process the glucose measurements 114 received from the computing device 106 and cause at least some of them to also be stored in the storage device 118. From the storage device 118, this data may be provided to, or otherwise accessed by, the malady identification system 122, e.g., to identify maladies among a population of users at a location, as described in more detail below.

In the illustrated example 300, the malady identification system 122 is depicted receiving the location data 120 and the temperature measurements 116 from the storage device 118. The malady identification system 122 is configured to use the location data 120 and the temperature measurements 116 to identify maladies at different locations. The malady identification system 122 is also depicted receiving the glucose measurements 114 in the illustrated example 300. In contrast to the location data 120 and the temperature measurements 116, though, the glucose measurements 114 that are depicted being communicated to the malady identification system 122 are illustrated with hashing. This represents that in one or more implementations, the malady identification system 122 identifies maladies among a population of users at a location without the glucose measurements 114 and that in other implementations, the malady identification system 122 does use the glucose measurements 114 to identify maladies among a population of users at a location. In accordance with the described techniques, the glucose measurements 114, the location data 120, and the temperature measurements 116 processed by the malady identification system 122 may correspond to one or more users of the user population 108, which may include the person 102.

In the illustrated example 300, the malady identification system 122 is depicted including identification logic 304. In general, the identification logic 304 is configured to process the temperature measurements 116 and the location data 120 to identify a malady in a population of users at one or more locations at some time, such as to identify the presence of a disease (e.g., influenza, coronavirus disease, etc.) among users located in a geographic region (e.g., a county) during a time interval (e.g., a last day, last week, etc.). In one or more implementations, the identification logic 304 may be configured to process the glucose measurements 114 along with the temperature measurements 116 and the location data 120 to identify a malady in a population of users at one or more locations at some time. In addition, the identification logic 304 is further usable to carry out this identification for multiple locations (e.g., multiple counties) and multiple times. Based on this identification, presence of a malady and/or its severity (e.g., number of cases of the malady or percentage of population having the malady) can be presented for different geographic regions (e.g., on a county to county basis) and/or for different times (e.g., on a day-to-day or week-to-week basis).

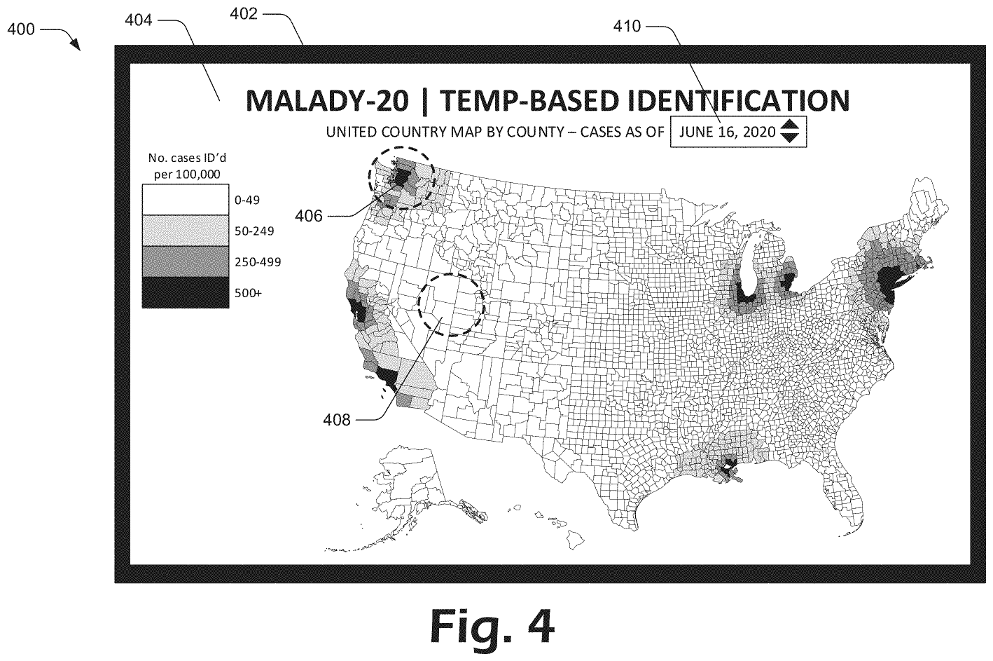

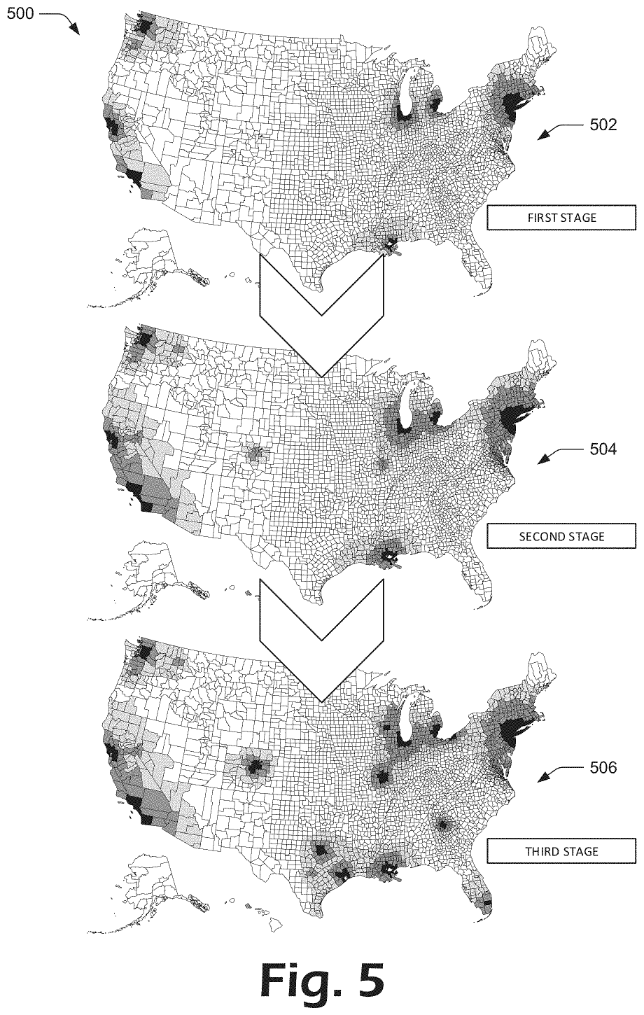

The identification also enables the identification logic 304 to compare the malady across different geographic regions and/or over time periods, such as by determining one or more statistical measures in relation to presence of the malady (e.g., differences) between different geographic regions or different time periods. By causing presentation of information that corresponds to the identification, the identification logic 304 enables users to compare (e.g., visually) presence of the malady across different geographic regions and/or over time periods. For example, the presence and/or severity of the malady at two different locations over a same time period (e.g., a last week) can be presented, as discussed in more detail in relation to at least FIG. 4. Alternately or additionally, the presence and/or severity of the malady at a given location can be compared across different periods of time, as discussed in more detail in relation to FIG. 5. As discussed above, the identification logic 304 may be configured to identify maladies for various types of geographic regions in accordance with the described techniques. Although counties are discussed above and below, for instance, the identification logic 304 may alternately or additionally identify maladies for countries, states, cities, zip codes, voting districts, and school districts, to name just a few.

To identify a malady among a population of users, the identification logic 304 may be configured in various ways without departing from the spirit or scope of the described techniques. For example, the identification logic 304 may include or be configured as a machine learning model or an ensemble of machine learning models, such as regression models (e.g., linear, polynomial, and/or logistic regression models), classifiers, neural networks, and reinforcement learning based models, to name just a few. Alternatively or additionally, the identification logic 304 may include or be configured as one or more hard-coded rules, such that the identification logic 304 processes the temperature measurements 116 and the location data 120 (and in some implementations additional data), for example, to determine one or more statistical measures related to malady identification. In this example, the identification logic 304 may then apply the above-mentioned rules to the one or more statistical measures. Additionally or alternatively, the identification logic 304 may be configured to detect anomalies in the glucose measurements 114 and/or the temperature measurements 116 in connection with the location data 120. In particular, the identification logic 304 can identify deviations from determined “normal” temperatures or glucose for a location. The deviations and the determined normal may correspond to average temperatures or glucose across the population of users located at a given location. It is to be appreciated that the identification logic 304 may be configured in other ways (e.g., based on various different algorithms and/or rules), to identify a malady among a population of users at a location during a given period of time, in accordance with the described techniques.

As noted above, the identification logic 304 may include or be configured as a machine learning model in or more implementations. In such implementations, the machine learning model may be generated according to one or more algorithms—to train the model to learn internal weights of the model (e.g., neural network approaches) or to learn parameters of a predictive function of the model (e.g., regression approaches)—and by using historical temperature measurements 116 and location data 120, e.g., of the user population 108. In implementations where additional data is used to identify a malady in a population of users, e.g., the glucose measurements 114, the machine learning model may be further trained using this additional data.

Along with historical temperature measurements 116 and location data 120, the machine learning model may also be generated using historical data describing the presence and/or absence of one or more maladies. Prior to generating the machine learning model, for instance, the historical temperature measurements 116 and historical location data 120 may be associated with data describing the presence or absence of a malady at a respective location and at a respective time. In other words, each historical temperature measurement 116 may be matched with a respective location (e.g., using the location data 120) and information describing whether at least one malady was present (or not) at the respective location at a time corresponding to the historical temperature measurement 116. In scenarios where historical glucose measurements 114 are also used by the identification logic 304 to identify maladies, each historical temperature measurement 116 may be further matched with one or more respective glucose measurements 114.

This matched data may be referred to as “training data” herein. The identification logic 304, when configured as a machine learning model, may be generated via a learning or training process performed according to one or more algorithms configured to learn function parameters from, or to train a model based on, this training data. In connection with this training or learning process, the historical temperature measurements 116 (and optionally the historical glucose measurements 114) may correspond to inputs to the identification logic 304 while the presence or absence of a malady at the respective location may correspond to a desired outcome, relative to which output of the identification logic 304 during the training or learning process may be compared. By using one or more of the learning or training algorithms noted above, the identification logic 304 learns to predict substantially the outcomes in the training data given the corresponding input training data. In particular, function parameters or internal model weights of the machine learning model are adjusted during the training process automatically, according to the learning or training algorithm employed, to cause predictions output by the model during the process to more closely match the outcomes of the training data. It is to be appreciated that a variety of learning approaches may be utilized in connection with the described techniques, including supervised approaches, unsupervised approaches, and reinforcement learning approaches.

In addition, the identification logic 304 may be trained using, and thus also be capable of receiving during operation, various other contextual information related to identification of a malady. By way of example, this contextual information may include a month, day of week, and time of day, to name just a few. The identification logic 304 can use this information, for instance, to inform detection of anomalies from expected temperature changes associated with nominal patterns. By way of example, an average temperature across a population may be lower at night, e.g., because the people in the population are sleeping and body temperature is lower during sleep. In this way, a relatively higher average temperature across the population during the night—which may not be notably (e.g., statistically significant) higher than the population's average temperature during the daytime—may indicate the presence of a malady among the population. Other examples of contextual information may include expected climate per month in different locations. It is to be appreciated that the identification logic 304 may be trained using, and also capable of leveraging during operation, a variety of contextual information without departing from the spirit or scope of the described techniques.

Once trained or parameters of an underlying function learned, the identification logic 304 configured as a machine learning model is configured for operation to identify maladies among a population of users at a location. More specifically, the identification logic 304 configured as a machine learning model identifies such a malady by generating a prediction regarding whether the malady is present at a particular location and over a time period, e.g., based on the training or learning. To do so, the temperature measurements 116 and the location data 120, and optionally the glucose measurements 114, are provided as input to the identification logic 304. Data describing other aspects about a user population may also be provided as input to the identification logic 304.

In one or more implementations, the malady identification system 122 preprocesses the location data 120 and the temperature measurements 116 to format them so they can be received as input by the identification logic 304. By way of example, the malady identification system 122 may form vectors (e.g., feature vectors) of the location data 120 and the temperature measurements 116, and then provide those vectors as input to the identification logic 304. In one or more implementations, the preprocessing may include determining statistical features of the location data 120 and the temperature measurements 116, including, for example, average temperature, average temperature over a time period, median temperature, number of users having temperatures above a threshold temperature, and percentage of users in a geographic region having temperatures above the threshold temperature, to name just a few. In these implementations, the malady identification system 122 may be configured to generate the input data to describe the determined statistical features in addition to or rather than describing the temperature measurements 116 and location data 120 directly. Based on patterns learned from the historical temperature measurements using the approaches discussed above, the identification logic 304 outputs a prediction regarding the presence of a malady at a given location over a particular time period based on the data input. The output may also be configured as a vector (e.g., a feature vector) indicative of the presence of the malady.

As noted above, the identification logic 304 may alternately or additionally be configured as one or more hard coded rules. Such rules may identify the presence or absence of a malady based on satisfaction of one or more criteria. By way of example, the rules may involve comparison to a threshold, such that if a threshold percentage of users in a geographic region have temperatures (or average temperatures) that surpass a threshold temperature over a time period, the identification logic 304 identifies the presence of a malady in the geographic region for the time period. It is to be appreciated that this is merely one example rule and that identification logic may encode a variety of rules to identify maladies among a population of users in a location in accordance with the described techniques.

Based on the data output by the identification logic 304, that describes one or more identified maladies, the malady identification system 122 may notify different entities. The illustrated example 300 includes notification 306 and notification 308. The notification 306 is illustrated being communicated to the computing device 106, which in one or more implementations is associated with the person 102, who corresponds to a user of the user population 108. Specifically, the person 102 may correspond to one user of the user population 108 that wears the wearable glucose monitoring device 104 and/or that has a user profile with the glucose monitoring platform 110. In contrast, the notification 308 is illustrated being communicated to third party 310. The third party 310 may correspond to a variety of entities that have an interest in maladies identified in a population of users by the malady identification system 122. By way of example, and not limitation, the third party 310 may represent a public health organization (e.g., the Center for Disease Control (CDC), the World Health Organization (WHO), and the National Institutes of Health (NIH)), a governmental organization, a school district, a health care facility (e.g., hospitals and doctors' offices), a news source, a telemedicine service, or a data partner (e.g., entities that have entered into an agreement with the glucose monitoring platform 110 to receive notifications of identified maladies), to name just a few.

In one or more implementations, the notifications 306, 308 comprise information describing a malady identified by the identification logic 304 in at least one geographic region for a time period, e.g., one or more previous time periods, a current time interval (e.g., today, this week, this month), one or more time periods subsequent to the identification (e.g., tomorrow, next week, next month). To this end, the notifications 306, 308 may include at least some of the same information. Alternately or additionally, the notifications 306, 308 may include different information. For instance, the notification 306 may comprise an alert or a warning directed to the person 102. Such an alert may include instructions recommending one or more behaviors for the user take based on identification of a malady at a location, such as a geographic region where the user is located or selected by the user. By way of example, the alert may inform the user of the malady and/or include suggestions of behaviors to mitigate the person 102's risk of contracting the malady, such as to wash hands more frequently than normal, avoid close interactions with other people, limit travel from home, wear a facial covering when around others, and so on. In at least one implementation, the notification 308 communicated to the third party 310 may not include alerts with such instructions, although in at least one different implementation the notification 308 may include some form of alert and/or recommended behaviors for mitigating an identified malady.

It is to be appreciated that the notifications 306, 308 may include the same information, or different information without departing from the spirit or scope of the techniques described herein. Regardless, the notifications 306, 308 include information based on and/or describing at least one malady identified by the identification logic 304 for a population of users in at least one geographic region over a time period. In the context of the different information that may be included in the notifications 306, 308 and then presented via the computing device 106, or a computing device associated with the third party 310, consider the following discussion of FIGS. 4-6.

FIG. 4 depicts an example 400 of an implementation of a user interface displayed for presenting information associated with population malady identification.

The illustrated example 400 includes display device 402 displaying user interface 404, which may be an example of the notifications 306, 308 or generated based on those notifications. In this example 400, the user interface 404 includes a display of a geographic region (e.g., a country) that is divided into further geographic regions (e.g., counties). Additionally, the user interface 404 includes graphical elements that visually indicate maladies identified in the further geographic regions. These visual elements are based on maladies identified by the identification logic 304 as discussed above.

The user interface 404 is one example of a “heat map” that may be generated based on maladies identified by the identification logic 304 among user populations in different areas over a time period. As a heat map, the user interface 404 is configured to show differences in the presence of a malady one location to another, such as to show “hot spots.” Hot spots correspond to locations where severity of the malady is identified as being relatively greater than other locations. In this example 400, location 406 is displayed with graphical elements that indicate a severity of a malady is greater than the severity indicated by graphical elements at different location 408. In this way, the presence or severity of the malady at two different locations can be visually indicated for a same time period. In other words, the heat map visually differentiates the severity of the malady across user populations at different locations depicted on the heat map.

In this example 400, a time associated with the one or more maladies in the displayed geographic regions is indicated by graphical time element 410. The graphical time element 410 may represent various time periods in accordance with the described techniques, such as a time period ranging from January 1 of a current year to an indicated date, a time period ranging from a selected data (e.g., a beginning of a “malady” season or first identified case of the malady) to an indicated date, a given day, a given week, a given month, a given year, and so forth.

FIG. 5 depicts an example 500 of an implementation of information presented in association with population malady identification via a user interface.

The illustrated example 500 depicts multiple stages of a geographic region (e.g., a country) that is divided into further geographic regions (e.g., counties), where the multiple stages include graphical elements that visually indicate maladies identified in the further geographic regions at different times of the multiple stages. In particular, the illustrated example 500 includes a first stage 502, a second stage 504, and a third stage 506, which may correspond to a first time, a second time, and a third time, respectively. In this example, the second time may be subsequent to the first time and the third time may be subsequent to the second time.

The stages 502, 504, 506 may be presented via a user interface, such as the user interface 404, and be examples of the notifications 306, 308 or generated based on those notifications. Each stage may correspond to a “heat map” indicative of a presence or severity of one or more maladies in the different locations at the corresponding time, e.g., at the first, second, or third time. In one or more implementations, the user interface 404 may be configured to display the different stages 502, 504, 506 in chronological and/or reverse chronological order and also animate transitions between those stages. In this way, the described systems can visually indicate to users how the presence and/or severity of one or more maladies changes over time. In this particular example 500, for instance, the map of the geographic region and the further geographic regions at the third stage 506 includes more hot spots than the map at the first stage 502, indicating an increased presence or severity of one or more maladies from the first time to the third time. In this example, the map at the second stage 504 indicates intermediate growth of the presence and/or severity of the maladies between the first stage 502 and the third stage 506. Generally speaking, presentation of a same geographic region or set of geographic regions at different times enables the presence and/or severity of a malady at a given location to be compared across the different times.

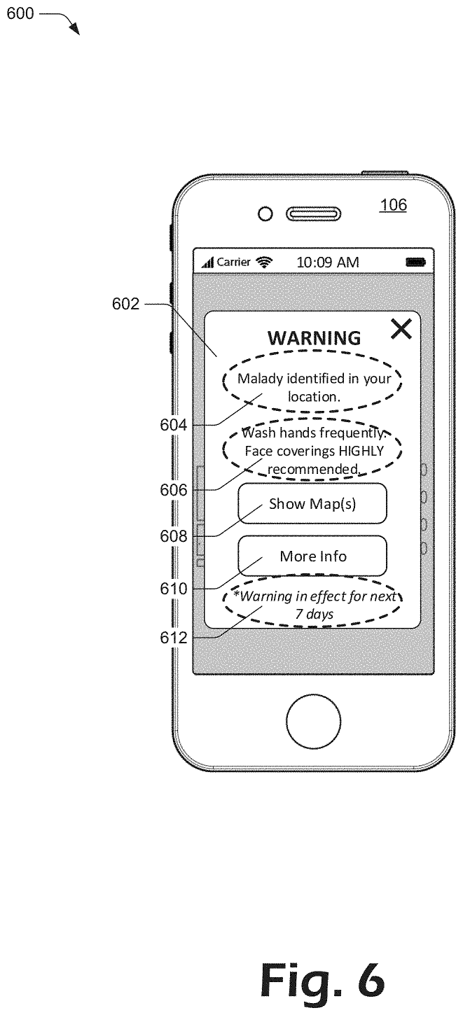

FIG. 6 depicts an example 600 of an implementation of a user interface displayed for presenting a notification associated with population malady identification.

The illustrated example 600 depicts one example of the computing device 106 displaying a user interface 602. The user interface 602 may be an example of the notification 306 or may be generated by the computing device 106 based on the notification 306. In this example 600, the user interface displays information 604, which describes a malady identified by the identification logic 304 as discussed above. Here, the user interface 602 also includes recommended behaviors 606. As discussed above, the recommended behaviors 606 may be suggested to mitigate a likelihood of a user of the computing device 106 contracting an identified malady.

In this example 600, the user interface 602 further includes selectable graphical elements 608, 610, which are selectable to display more information about one or more maladies identified by the identification logic 304, namely, a map that visually indicates presence or severity of the malady (e.g., as in FIGS. 4 and 5) and additional information. The exemplary user interface 602 also includes a warning expiration 612. It is to be appreciated that the user interface 602 is one example of an alert that may be displayed based on the notification 306. Alerts may be configured for display in different ways to include different and/or additional information without departing from the spirit or scope of the described techniques. Alternately or additionally, a warning may be displayed as a notification from a mobile app, within the mobile app, received and displayed as text message, and so forth.

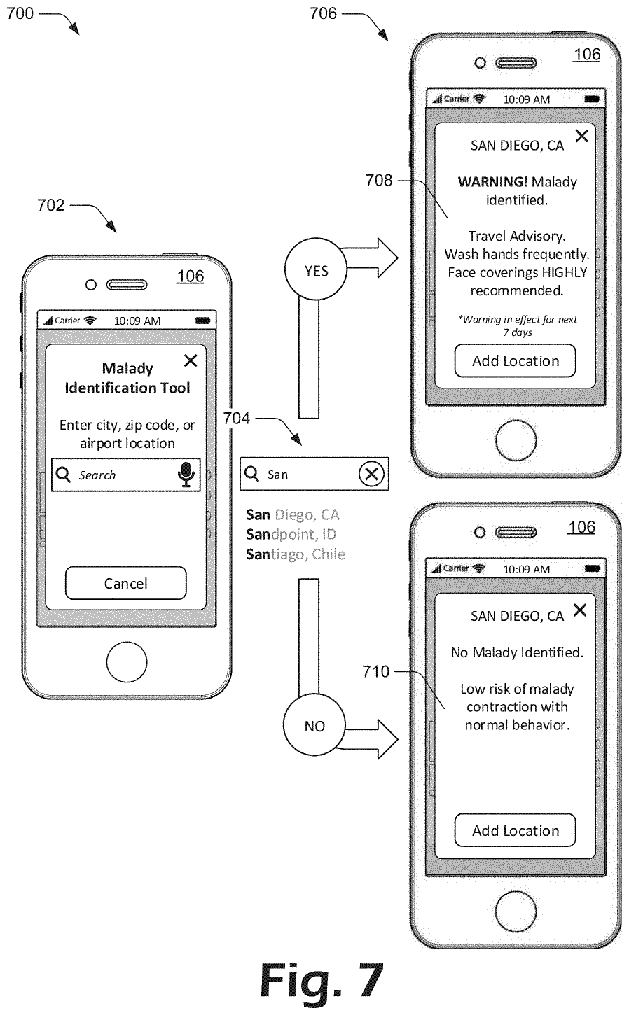

FIG. 7 depicts an example 700 of an implementation of user interfaces displayed for presenting information associated with population malady identification at a selected location.

The illustrated example 700 includes the computing device 106 in an example implementation where a user of the computing device 106 selects a location, the identification logic 304 identifies whether a malady is detected in the selected location based on the temperature measurements 116, and the computing device 106 displays an indication in relation to the identification, such as an indication that a malady is present in the location or an indication that a malady is not present in the location.

At first stage 702, for instance, the computing device 106 presents a user interface that allows the user to provide input to select a geographic region. The depicted interface indicates that the user may begin typing in characters of a place name or speak a place name. At second stage 704, the computing device 106 presents suggested geographic regions that match a portion of a search query input by the user for selecting a geographic region. Although not depicted in the illustrated example 700, the user of the computing device 106 selects a location, i.e., San Diego, Calif.

At stage 706 the computing device 106 presents an indication of whether a malady is identified by the identification logic 304 at the selected location or not. In particular, user interface configuration 708 corresponds to a scenario where the identification logic 304 does identify a malady (“YES”) at the selected geographic region and/or predicts there is a risk of the malady at the region over an upcoming time period. In contrast, user interface configuration 710 corresponds to a scenario where the identification logic 304 does not identify a malady (“NO”) at the selected geographic region and/or predicts there is not a risk of the malady at the region over an upcoming time period.

Having discussed example details of the techniques for population malady identification with a wearable glucose monitoring device, consider now some example procedures to illustrate additional aspects of the techniques.

### Example Procedures

This section describes example procedures for population malady identification with a wearable glucose monitoring device. Aspects of the procedures may be implemented in hardware, firmware, or software, or a combination thereof. The procedures are shown as a set of blocks that specify operations performed by one or more devices and are not necessarily limited to the orders shown for performing the operations by the respective blocks. In at least some implementations the procedures are performed by a malady identification system, such as the malady identification system 122 that makes use of the identification logic 304.

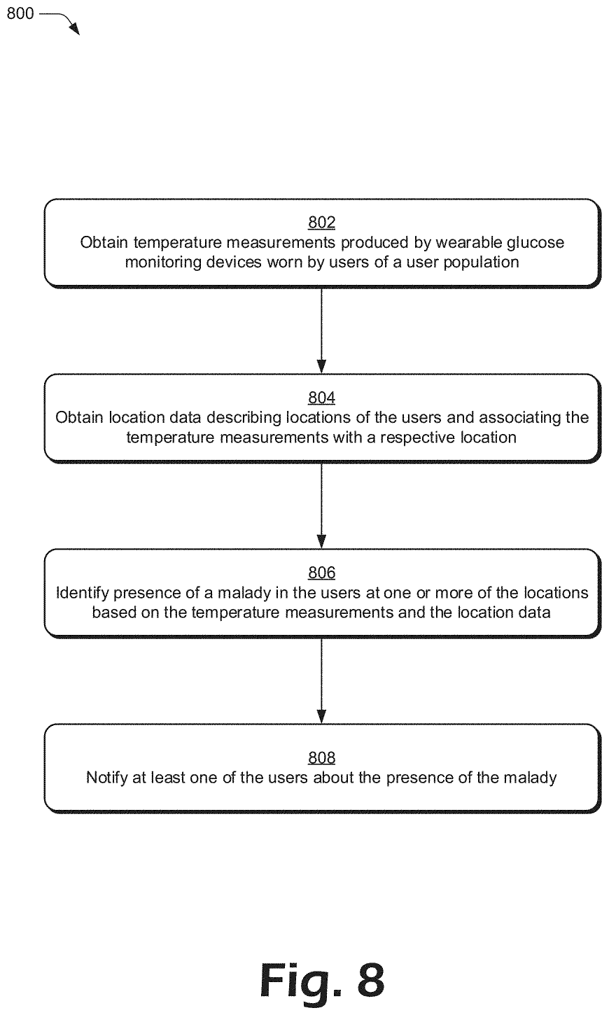

FIG. 8 depicts a procedure 800 in an example implementation in which presence of a malady is identified in users at one or more locations based on temperature measurements obtained from wearable glucose monitoring devices.

Temperature measurements are obtained that are produced by wearable glucose monitoring devices worn by users of a user population (block 802). By way of example, the malady identification system 122 obtains the temperature measurements 116 from the storage device 118 of the glucose monitoring platform 110. The temperature measurements 116 are produced by wearable glucose monitoring devices 104 of users of the user population 108.

Location data describing locations of the users is obtained and the temperature measurements are associated with a respective location according to the location data (block 804). By way of example, the malady identification system 122 obtains the location data 120. In one or more implementations, computing devices 106 associated with users of the user population 108 associate the location data 120 with the respective temperature measurements 116, such that the malady identification system 122 is able to associate locations described by the location data 120 with each of the temperature measurements 116. Alternately or additionally, the storage device 118 maintains the location data 120 as part of user profiles for users of the user population 108. Here, the malady identification system 122 is able to associate locations described by the location data 120 of the user profiles for the users of the user population 108 with each of the temperature measurements 116.

Presence of a malady is identified in the users at one or more locations based on the temperature measurements and the location data (block 806). By way of example, the identification logic 304 identifies a presence of a malady in the users at one or more of the locations based on the temperature measurements 116 and the location data 120. In one or more implementations, the identification logic 304 identifies the presence of the malady further based on the glucose measurements 114.

At least one of the users is notified about the presence of the malady (block 808). By way of example, the malady identification system 122 communicates the notification 306 to the computing device 106 to inform the person 102 about the presence of the malady identified at block 806. The notification 306 may include or otherwise enable presentation, via the computing device 106, of one or more of user interfaces to output information about the identified malady, such as one or more of the user interfaces depicted in FIGS. 4-7.

In one or more implementations, at least one third party is notified about the presence of the malady. By way of example, the malady identification system 122 communicates the notification 308 to the third party 310 to inform at least one user associated with the third party 310 about the malady identified at block 806. The notification 308 may include or otherwise enable presentation, via a computing device, of one or more user interfaces to output information about the identified malady, such as one or more of the user interfaces depicted in FIGS. 4-7.

Having described example procedures in accordance with one or more implementations, consider now an example system and device that can be utilized to implement the various techniques described herein.

### Example System and Device

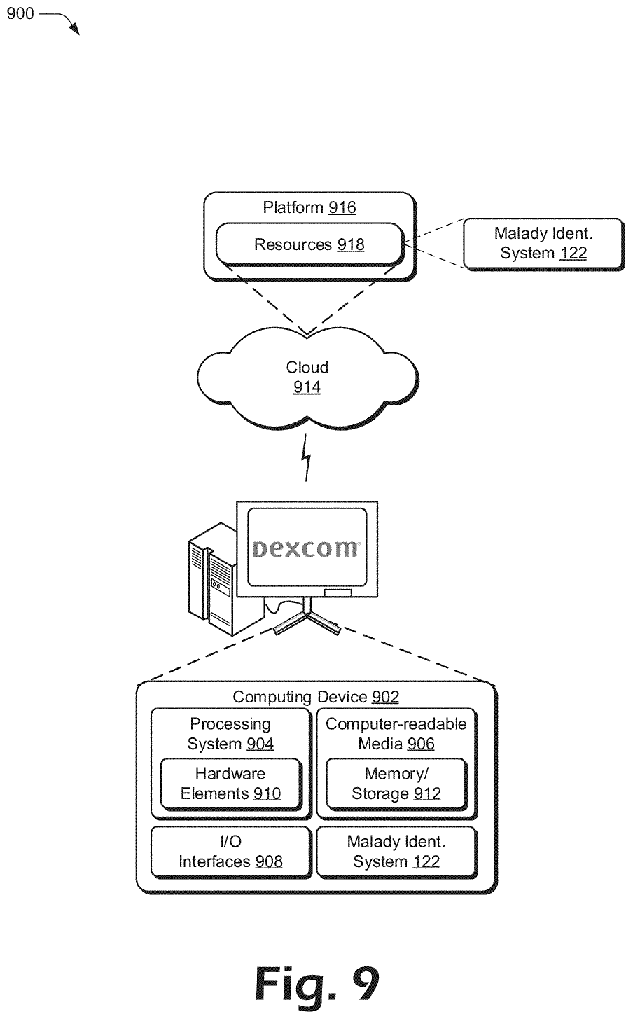

FIG. 9 illustrates an example system generally at 900 that includes an example computing device 902 that is representative of one or more computing systems and/or devices that may implement the various techniques described herein. This is illustrated through inclusion of the malady identification system 122. Here, the malady identification system 122 is illustrated both at the computing device 902 level and a service provider level. This indicates that some of the aspects of the malady identification system 122 may be implemented at the computing device 902 (e.g., the computing device 106), such as in connection with a malady identification app. This also indicates that aspects of the malady identification system 122 are implemented using one or more server-based or “cloud computing” resources. The computing device 902 may be, for example, a server of a service provider, a device associated with a client (e.g., a client device), an on-chip system, and/or any other suitable computing device or computing system.

The example computing device 902 as illustrated includes a processing system 904, one or more computer-readable media 906, and one or more I/O interfaces 908 that are communicatively coupled, one to another. Although not shown, the computing device 902 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 904 is representative of functionality to perform one or more operations using hardware. Accordingly, the processing system 904 is illustrated as including hardware elements 910 that may be configured as processors, functional blocks, and so forth. This may include implementation in hardware as an application specific integrated circuit or other logic device formed using one or more semiconductors. The hardware elements 910 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 906 is illustrated as including memory/storage 912. The memory/storage 912 represents memory/storage capacity associated with one or more computer-readable media. The memory/storage 912 component may include volatile media (such as random access memory (RAM)) and/or nonvolatile media (such as read only memory (ROM), Flash memory, optical disks, magnetic disks, and so forth). The memory/storage 912 component may include fixed media (e.g., RAM, ROM, a fixed hard drive, and so on) as well as removable media (e.g., Flash memory, a removable hard drive, an optical disc, and so forth). The computer-readable media 906 may be configured in a variety of other ways as further described below.

Input/output interface(s) 908 are representative of functionality to allow a user to enter commands and information to computing device 902, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 902 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 902. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage thereon of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 902, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 910 and computer-readable media 906 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 910. The computing device 902 may be configured to implement particular instructions and/or functions corresponding to the software and/or hardware modules. Accordingly, implementation of a module that is executable by the computing device 902 as software may be achieved at least partially in hardware, e.g., through use of computer-readable storage media and/or hardware elements 910 of the processing system 904. The instructions and/or functions may be executable/operable by one or more articles of manufacture (for example, one or more computing devices 902 and/or processing systems 904) to implement techniques, modules, and examples described herein.

The techniques described herein may be supported by various configurations of the computing device 902 and are not limited to the specific examples of the techniques described herein. This functionality may also be implemented all or in part through use of a distributed system, such as over a “cloud” 914 via a platform 916 as described below.

The cloud 914 includes and/or is representative of a platform 916 for resources 918. The platform 916 abstracts underlying functionality of hardware (e.g., servers) and software resources of the cloud 914. The resources 918 may include applications and/or data that can be utilized while computer processing is executed on servers that are remote from the computing device 902. Resources 918 can also include services provided over the Internet and/or through a subscriber network, such as a cellular or Wi-Fi network.

The platform 916 may abstract resources and functions to connect the computing device 902 with other computing devices. The platform 916 may also serve to abstract scaling of resources to provide a corresponding level of scale to encountered demand for the resources 918 that are implemented via the platform 916. Accordingly, in an interconnected device embodiment, implementation of functionality described herein may be distributed throughout the system 900. For example, the functionality may be implemented in part on the computing device 902 as well as via the platform 916 that abstracts the functionality of the cloud 914.

### CONCLUSION

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A method comprising:
obtaining temperature measurements produced by wearable glucose monitoring devices worn by users of a user population;
obtaining location data describing locations of the users and associating each of the temperature measurements with a respective location;
identifying presence of a malady in the users at one or more of the locations based on the temperature measurements and the location data; and
notifying at least one of the users about the presence of the malady.

2. The method of claim 1, wherein identifying the presence of the malady includes processing the temperature measurements and the location data, in part, using one or more machine learning models, the one or more machine learning models generated based on historical temperature measurements of the user population and historical data describing presence of one or more maladies in the user population.

3. The method of claim 1, wherein the wearable glucose monitoring devices include at least one continuous glucose monitoring (CGM) system.

4. The method of claim 1, wherein the identifying is further based on glucose measurements produced by the wearable glucose monitoring devices worn by the users of the user population.

5. The method of claim 4, wherein identifying the presence of the malady includes processing the temperature measurements, the glucose measurements, and the location data, in part, using one or more machine learning models, the one or more machine learning models generated based on historical temperature and glucose measurements of the user population and historical data describing presence of one or more maladies in the user population.

6. The method of claim 1, further comprising notifying at least one third party about the presence of the malady.

7. The method of claim 6, wherein the at least one third party includes at least one of: a public health organization, a governmental organization, a school district, a health care facility, a news source, a telemedicine service, or a data partner with a glucose monitoring platform that corresponds to the wearable glucose monitoring devices.

8. The method of claim 1, wherein the users of the user population have user profiles with a glucose monitoring platform.

9. The method of claim 1, wherein notifying at least one user about the presence of the malady includes:
generating a heat map that visually differentiates severity of the malady across user populations at different locations; and
causing display of the heat map on a display device of a computing device associated with the at least one user.

10. The method of claim 1, wherein notifying at least one user about the presence of the malady includes:
generating an alert having information about the presence of the malady; and
causing output of the alert via a computing device associated with the at least one user.

11. The method of claim 10, wherein causing output of the alert comprises causing display of the alert via a display device of the computing device.

12. A system comprising:
at least one processor; and
memory having instructions stored thereon that are executable by the at least one processor to perform operations including:
obtaining temperature measurements produced by wearable glucose monitoring devices worn by users of a user population;
obtaining location data describing locations of the users and associating the temperature measurements with a respective location;
identifying presence of a malady in the users at one or more of the locations based on the temperature measurements and the location data; and
notifying at least one of the users about the presence of the malady.

13. The system of claim 12, further comprising one or more machine learning models configured to identify the presence of the malady by processing the temperature measurements and the location data, the one or more machine learning models generated based on historical temperature measurements of the user population and historical data describing presence of one or more maladies in the user population.

14. The system of claim 12, wherein the wearable glucose monitoring devices include at least one continuous glucose monitoring (CGM) system.

15. The system of claim 12, wherein the identifying is further based on glucose measurements produced by the wearable glucose monitoring devices worn by the users of the user population.

16. The system of claim 15, further comprising one or more machine learning models configured to identify the presence of the malady by processing the temperature measurements, the glucose measurements, and the location data, the one or more machine learning models generated based on historical temperature and glucose measurements of the user population and historical data describing presence of one or more maladies in the user population.

17. The system of claim 12, wherein the operations further include notifying at least one third party about the presence of the malady.

18. The system of claim 17, wherein the at least one third party includes at least one of: a public health organization, a governmental organization, a school district, a health care facility, a news source, a telemedicine service, or a data partner with a glucose monitoring platform that corresponds to the wearable glucose monitoring devices.

19. The system of claim 12, wherein the users of the user population have user profiles with a glucose monitoring platform.

20. The system of claim 12, wherein notifying at least one user about the presence of the malady includes:
generating a heat map that visually differentiates severity of the malady across user populations at different locations; and
causing display of the heat map on a display device of a computing device associated with the at least one user.

21. The system of claim 12, wherein notifying at least one user about the presence of the malady includes:
generating an alert having information about the presence of the malady; and
causing output of the alert via a computing device associated with the at least one user.

22. The system of claim 21, wherein causing output of the alert comprises causing display of the alert via a display device of the computing device.

23. One or more non-transitory computer-readable storage media having instructions stored thereon that are executable by one or more processors of at least one computing device to cause the at least one computing device to perform operations comprising:
obtaining temperature measurements produced by wearable glucose monitoring devices worn by users of a user population;
obtaining location data describing locations of the users and associating the temperature measurements with a respective location;
identifying presence of a malady in the users at one or more of the locations based on the temperature measurements and the location data; and
notifying at least one of the users about the presence of the malady.

24. The one or more computer-readable storage media of claim 23, wherein identifying the presence of the malady includes processing the temperature measurements and the location data, in part, using one or more machine learning models, the one or more machine learning models generated based on historical temperature measurements of the user population and historical data describing presence of one or more maladies in the user population.

25. The one or more computer-readable storage media of claim 23, wherein the wearable glucose monitoring devices include at least one continuous glucose monitoring (CGM) system.

26. The one or more computer-readable storage media of claim 23, wherein the identifying is further based on glucose measurements produced by the wearable glucose monitoring devices worn by the users of the user population.

27. The one or more computer-readable storage media of claim 26, wherein identifying the presence of the malady includes processing the temperature measurements, the glucose measurements, and the location data, in part, using one or more machine learning models, the one or more machine learning models generated based on historical temperature and glucose measurements of the user population and historical data describing presence of one or more maladies in the user population.

28. The one or more computer-readable storage media of claim 23, wherein the operations further include notifying at least one third party about the presence of the malady.

29. The one or more computer-readable storage media of claim 28, wherein the at least one third party includes at least one of: a public health organization, a governmental organization, a school district, a health care facility, a news source, a telemedicine service, or a data partner with a glucose monitoring platform that corresponds to the wearable glucose monitoring devices.

30. The one or more computer-readable storage media of claim 23, wherein the users of the user population have user profiles with a glucose monitoring platform.

31. The one or more computer-readable storage media of claim 23, wherein notifying at least one user about the presence of the malady includes:
generating a heat map that visually differentiates severity of the malady across user populations at different locations; and
causing display of the heat map on a display device of a computing device associated with the at least one user.

32. The one or more computer-readable storage media of claim 23, wherein notifying at least one user about the presence of the malady includes:
generating an alert having information about the presence of the malady; and
causing output of the alert via a computing device associated with the at least one user.

33. The one or more computer-readable storage media of claim 32, wherein causing output of the alert comprises causing display of the alert via a display device of the computing device.

34. An apparatus comprising:
means for obtaining temperature measurements produced by wearable glucose monitoring devices worn by users of a user population;
means for obtaining location data describing locations of the users and associating each of the temperature measurements with a respective location;
means for identifying presence of a malady in the users at one or more of the locations based on the temperature measurements and the location data; and
means for notifying at least one of the users about the presence of the malady.
